(19) United States
(12) Patent Application Publication
(10) Pub. No.: US 2010/0087007 A1
(43) Pub. Date: Apr. 8, 2010

(54) METHOD FOR QUANTITATIVE DETERMINATION OF NICKEL AND/OR COPPER AND EQUIPMENT TO BE USED IN THE METHOD

(75) Inventors: Hitoshi Mizuguchi, Kanagawa-ken (JP); Junichi Shida, Kanagawa-ken (JP); Masamitsu Iiyama, Kanagawa-ken (JP); Mitsugu Abe, Kanagawa-ken (JP)

Correspondence Address: FOLEY AND LARDNER LLP;SUITE 500, 3000 K STREET NW, WASHINGTON, DC, 20007, US

(73) Assignee: Nomura Micro Science Co. Ltd

(21) Appl. No.: 12/516,462

(22) PCT Filed: Nov. 26, 2007
(86) PCT No.: PCT/JP2007/072778

§ 371 Date: May 27, 2009

(30) Foreign Application Priority Data
Nov. 28, 2006 (JP) 2006 319609

(51) Int. Cl. G01N 33/20; B01J 8/00

(52) U.S. Cl. 436/84; 436/80; 422/187

(57) ABSTRACT

A method for quantitative determination of nickel and/or copper, by which an ultratrace amount of nickel and/or copper contained in a liquid sample can be easily and simply determined in situ; and apparatus to be used in the method. The method comprises a step of adding a complex-forming agent capable of forming a complex with nickel and copper to a liquid sample containing nickel and/or copper in unknown concentrations to form colored fine particles of a nickel complex and/or a copper complex and a step of determining the quantities of nickel and/or copper on the basis of the colored fine particles.

### TECHNICAL FIELD

The present invention relates to a method for a simple determination of ultra-trace nickel and/or copper in liquid samples and an apparatus used for the method.

### BACKGROUND ART

Conventionally, Nickel and copper determination techniques, including inductively coupled plasma with atomic emission spectrometry or mass spectrometry, and graphite furnace atomic absorption spectrometry are highly developed. However, these methods need expensive instrumentations with special laboratory skills and are not suitable for on-site analysis.

For the determination of trace nickel, it is well known that absorption spectroscopy followed by solvent extraction with dimethylglyoxime (see, for example, JIS G1216) is available. However, this method, in which organic solvents harmful to human body are used as an extracting solvent, has many disadvantages from a practical view point and also has insufficient sensitivity for the determination at parts per billion levels. And, for the determination of trace copper, it is well known that absorption spectroscopy followed by solvent extraction with diethyldithiocarbamate (see JIS K0102) is available. However, this method also has insufficient sensitivity for the determination at parts per billion levels.

Therefore, there is not an appropriate method for measurement requiring the detection of a minute amount of ions at the ppb level, for example, in a semiconductor polishing slurry and a semiconductor washing chemical solution used for a semiconductor device production process. There is a demand for a method capable of quantitative determination of nickel and copper, which are present in a minute amount in the above solution, on site.

Reference 1: JIS G1216

Reference 2: JIS K0102

### DISCLOSURE OF THE INVENTION

The present invention provides a method for quantitative determination of nickel and/or copper, by which an ultratrace amount of nickel and/or copper contained in a liquid sample can be easily and simply determined in situ; and an apparatus to be used for the method.

The method for quantitative determination of nickel and/or copper of the invention comprises a step of adding a complex-forming agent capable of forming a complex with nickel and copper to a liquid sample containing nickel and/or copper in unknown concentrations to form colored fine particles of a nickel complex and/or a copper complex and a step of determining the quantities of nickel and/or copper on the basis of the colored fine particles of the nickel complex and/or copper complex.

A quantitative determination apparatus for nickel and/or copper of the present invention is provided with a sample pipe to collect a liquid sample from a liquid sample line including unknown concentrations of nickel and/or copper at predetermined time intervals, a complex-forming agent storing tank to store a complex-forming agent which forms a complex with the nickel and copper contained in the liquid sample, a reaction vessel to form colored fine particles of the nickel complex and/or copper complex by reacting the complex-forming agent to be fed from the complex-forming agent storing tank and the liquid sample to be fed from the sample pipe, and a quantitative determination means to quantify the colored fine particles of the nickel complex and/or copper complex to be fed from the reaction vessel.

Here, prior to the step of forming the colored fine particles of the nickel complex and/or copper complex, at least one pretreatment step selected from a step of prefiltering a liquid sample, a step of adding a salting-out agent to the liquid sample, a step of adding a pH adjuster and a step of adding a masking agent may be performed if necessary. In a case where the plural pretreatment steps are performed, the order of the pretreatment steps is not limited to a particular one if the other steps are not disturbed.

To perform the pretreatment steps, it is appropriate for the quantitative determination apparatus to provide each step with a corresponding chemical agent tank and a mixing vessel to mix the chemical agent fed from the chemical agent tank with a liquid sample. For example, when it is assumed that the apparatus performs all the pretreatment steps, it may be provided with a tank to add a salting-out agent to the liquid sample, a first reaction vessel to react the chemical solution fed from the salting-out agent tank with the liquid sample, a pH adjuster tank to store a chemical solution for adjusting the pH value of the liquid sample, a second reaction vessel to react the chemical solution fed from the pH adjuster tank with the liquid sample fed from the first reaction vessel, a masking agent tank to store a masking agent for suppressing interference by coexisting metals other than nickel and copper in the pH-adjusted liquid sample, a third reaction vessel to react the masking agent fed from the masking agent tank with the liquid sample fed from the second reaction vessel, a complex-forming agent storing tank to store a complex-forming agent for forming a complex with nickel and copper in the masking agent-added liquid sample, a fourth reaction vessel to form colored fine particles of a nickel complex and/or a copper complex by reacting the complex-forming agent fed from the complex-forming agent storing tank with the liquid sample fed from the third reaction vessel, and a quantitative determination means to quantify the colored fine particles of the nickel complex and/or copper complex fed from the fourth reaction vessel. It is preferable to provide a filter having a filtration membrane for capturing the nickel complex and/or copper complex before the quantitative determination means. The quantitative determination means may be configured to quantify the nickel and/or copper complex which is captured by the filtration membrane.

By configuring as described above, an ultratrace amount of nickel and/or copper contained in the liquid sample can be quantified simply and easily on site.

Embodiments of the invention are described below with reference to the drawings.

First, it is preferable to remove fine particles in advance from a liquid sample containing unknown concentrations of nickel and/or copper to selectively separate nickel and/or copper avoiding interference by coexisting materials. A filter used to remove the fine particles has a pore diameter which is normally in a range of 0.015 to 12 μm, and preferably in a range of 0.015 to 3.0 μm. It is also preferable that the liquid sample is adjusted to have an appropriate pH, which is normally in a range of 6 to 14, and preferably in a range of 7 to 12. For the pH adjustment, a chemical solution such as an acidic solution, an alkaline solution, a buffering agent or the like is used. As the buffering agent, it is preferable to use ammonium salt, an amino acid such as glycine or sarcosine, amines, borax, borate salt, phosphoric salt, a tris buffering agent, Good's buffer or the like. If the pH of the liquid sample is not in a preferable pH range, it is necessary to perform a neutralization treatment, when the liquid sample is acidic, it is preferable to neutralize by hydroxide of alkali metal, and when the liquid sample is alkaline, it is preferable to neutralize by an inorganic acid. Especially, nitric acid or perchloric acid which has high solubility of salt and is hard to form a complex with impure metal is preferable.

When the liquid sample is, for example, a semiconductor washing chemical solution or a semiconductor polishing slurry used in a semiconductor device production process, it is necessary to remove together with abrasive grains, components of the chemical solution and the like, and it is preferable to extract the contained nickel and/or copper by solid-phase extraction or solvent extraction. The solid-phase extraction method preferably uses, for example, an ion exchange resin or a chelating resin. For the solvent extraction method, dimethylglyoxime and its derivative, diphenylthiocarbazone (dithizone) and its derivative, β-diketones, 8-quinolinol (oxine) and its derivative, diethyldithiocarbamate and its analog and the like are preferable as a ligand. As the extraction solvent, a solvent such as chloroform, carbon tetrachloride, benzene, nitrobenzene, toluene, hexane, methyl isobutyl ketone or the like, which forms two layers when mixed with water, or a mixed solution thereof, or a mixed solution containing acetone and ethanol is preferable. As a back-extraction agent, hydrochloric acid, nitric acid, sulfuric acid, perchloric acid or the like is preferable.

Then, a masking agent is added to the treated liquid sample to suppress interference by coexisting metals other than nickel and/or copper in the liquid sample. As the masking agent, for example, organic carboxylate such as citric salt or salt of tartaric acid, thiosulfate, ammonium salt, cyanide, sulfide, ethylenediamine, fluoride, iodide, triethanolamine, and amino polycarboxylate such as ethylenediaminetetraacetate or the like is used, and sodium thiosulfate is preferable.

The salting-out agent is added to the liquid sample to promote formation of fine particles of a nickel and copper complex dissolved in the liquid sample. As the salting-out agent, for example, alkali metal salt or alkaline earth metal salt such as sodium chloride, sodium nitrate or the like is used and are not particularly limited if it does not form a complex with nickel and/or copper to be measured or does not produce particles other than the target fine particles of the nickel and copper complex in the liquid sample.

Subsequently, a complex-forming agent is added to the liquid sample to produce colored fine particles of the nickel complex and/or copper complex. As the complex-forming agent, for example, an oxime compound, an azo compound or the like is used if it reacts with nickel and copper to form complexes of nickel and copper, and α-furildioxime is preferably used.

A liquid sample containing the colored fine particles of the nickel complex and/or copper complex is passed through a filtration membrane to collect colored compounds of the nickel complex and/or copper complex on the filtration membrane, thereby separating and condensing them, and the filtration membrane is colored. The liquid sample having colored the filtration membrane is drained. As the filtration membrane, a reverse osmosis membrane, an ultrafiltration membrane and a microfiltration membrane are usable, and they have a pore diameter in a range of 0.015 to 12 μm, and preferably in a range of 0.015 to 3.0 μm. And, since the nickel complex and copper complex are selectively adsorbed, the used material is, for example, cellulose acetate, nitrocellulose, polycarbonate, polyethylene, polypropylene, polyvinyl alcohol, polytetrafluoroethylene (PTFE) or the like. The membrane thickness is not particularly limited but normally selected from a range of 6 μm to 1 mm. A flow rate is not particularly limited, but it is preferably 0.3 ml/sec or below (but, an effective filtration area of about 120 mm²) for quantitative adsorption of nickel and copper.

Concentrations of nickel and/or copper can be determined by performing colorimetry of the levels of color tone and color density of the colored filtration membrane. The colored color density can be subject to the colorimetry visually according to a color tone comparison table but can also be quantified by digitalizing by measuring equipment such as a spectrophotometer.

As described above, the method for quantitative determination of nickel and/or copper of this embodiment extracts nickel and/or copper from the liquid sample containing nickel and/or copper by solid-phase extraction or solvent extraction, adds a masking agent and a complex-forming agent sequentially, mixes them, and determines concentrations of nickel and/or copper from the produced colored fine particles of the nickel complex and/or copper complex. Thus, nickel and/or copper present in an ultratrace amount at the ppb level in the liquid sample, for example, a semiconductor polishing slurry can be easily and simply quantified on site. Since the fine particles of nickel and/or copper can be condensed on the filtration membrane, the quantitative determination can be made with high sensitivity in comparison with a conventional method of quantifying the liquid sample by absorption spectroscopy.

In this embodiment, the complex-forming agent is added to the liquid sample to form the colored fine particles of the nickel complex and/or copper complex, which is then passed through the filtration membrane. Color densities of the colored filtration membrane are subjected to colorimetry to determine the concentrations of nickel and/or copper. But nickel and/or copper can also be quantified from light scattering intensity without passing through the filtration membrane. The light scattering intensity measuring method irradiates laser light or white light, detects light scattered from the fine particles of the nickel complex and/or copper complex, converts into electric signals and measures the number of fine particles. As the light scattering intensity measuring equipment used for measuring is, for example, a multi-angle particle diameter analysis system DLS-7000 manufacture by Otsuka Electronics Co., Ltd.

In this embodiment, the liquid sample exemplified includes a semiconductor polishing slurry, a semiconductor washing chemical solution and the like, but clean water or drinking water such as tap water, well water or the like can also be applied.

The quantitative determination apparatus for nickel and/or copper of this embodiment is described below. FIG. 15 is a diagram schematically showing an example of the structure to describe the quantitative determination apparatus for nickel and/or copper of the embodiment. In this embodiment, the liquid sample containing nickel and/or copper is passed through the filtration membrane, and FIA (Flow Injection Analysis) can be used to mix and react a buffering agent, a masking agent and a complex-forming agent continuously and efficiently.

The quantitative determination apparatus for nickel and/or copper comprises a sample pipe 2 to collect a liquid sample from a liquid sample line 1 through which the liquid sample containing unknown concentrations of nickel and/or copper flows, a salting-out agent tank 3 to store a salting-out agent, a pH adjuster tank 4 to store a chemical solution for pH adjustment, a masking agent tank 5 to store a masking agent, a complex-forming agent tank 6 to store a complex-forming agent, a first reaction vessel 7 to react the liquid sample and the salting-out agent by mixing them, a second reaction vessel 8 to react the liquid sample and the pH adjuster by mixing them, a third reaction vessel 9 to react the liquid sample and the masking agent by mixing them, a fourth reaction vessel 10 to react by additionally mixing the complex-forming agent, a filter 11 to filter the reaction solution fed from the fourth reaction vessel 10, a detector 12 to detect the colored degree of the filtration membrane surface, and a discharge line 17 to discharge the filtrate. The pipes to send the salting-out agent, the pH adjuster, the masking agent and the complex-forming agent to the first reaction vessel 7, the second reaction vessel 8, the third reaction vessel 9 and the fourth reaction vessel 10 each have pumps 13, 14, 15, 16. A filter to remove impure fine particles, a chemical solution tank to store an acid or alkali solution to adjust a pH, a solid-phase extraction column or a solvent extraction column may be mounted in front of the reaction vessel depending on a type of liquid sample, and a light scattering intensity meter may be used instead of the filter 11 or the spectrophotometer.

The liquid sample containing unknown concentrations of nickel and copper is passed through the sample pipe 2 at prescribed time intervals from the liquid sample line 1 as shown in FIG. 15 to collect a prescribed amount of liquid sample. The pH of the collected liquid sample is adjusted within the above-described condition range by mixing the pH adjuster fed from the pH adjuster tank 3 by means of the pump 12 and the inflow liquid sample by the first reaction vessel 6. The pH-adjusted liquid sample is mixed with the masking agent fed from the masking agent tank 4 by the pump 13 by the second reaction vessel 7 to suppress interference by the coexisting metals.

Subsequently, the solution fed from the complex-forming agent tank 5 by the pump 14 and the liquid sample are reacted by mixing in the third reaction vessel 8 to produce the colored fine particles of the nickel complex and/or copper complex.

The liquid sample containing the colored fine particles of the nickel complex and/or copper complex is forwarded to the filter 9 and passed through the filtration membrane to selectively adsorb the colored compounds of the nickel complex and/or copper complex for separation and condensation. Thus, the fine particles are captured on the filtration membrane. The liquid sample having passed through the filtration membrane is discharged from a line 11.

Depending on the levels of color tone and color density of the filtration membrane which has captured and colored the fine particles, concentrations of nickel and/or copper are determined by the detector 10. The colored color density can be subject to colorimetry visually according to a color tone comparison table but can also be quantified by digitalizing from the absorption of visible light, ultraviolet light, fluorescent light by using a spectrophotometer. The produced fine particles can be quantified for nickel and/or copper from the number of fine particles and particle diameter of nickel and/or copper by light scattering intensity measurement.

The filter 11, the detector 12 and the line 17 shown in FIG. 15 are described below in detail with reference to FIGS. 16 to 19.

A filter roll 18A has a shape shown in FIG. 18, and a filter sheet which is covered with a double-sided tape is wound around a portion other than the filter portions 18a of the filter (filtration membrane). The double-sided tape is bored to have circular holes, and the portions other than the filter portions 18a are not contacted to the liquid and serve to prevent the liquid sample from leaking. The filter roll 18A is designed to forward the filter automatically, and the filter is wound up by a filter roll 18B every time the filter is forwarded to keep supplying new filter portions 18a.

The liquid sample supplied from the flow of FIG. 15 passes through a liquid sample introduction port 19 and filtered by the filter portion 18a of the filter. By rotating, the sample introduction port 19 forwards the filter, so that the next filter portion is aligned with the next sample introduction port and the next liquid sample is filtered.

The liquid sample passes through a liquid sample discharge funnel 20 and drained into a drainage tank 21. If necessary, the drainage is discharged through a drainage line 22.

The filtering step is performed by suction filtration using a vacuum pump 23, and a suction force is adjusted by means of a cock 24.

The liquid sample is filtered, and the amount of fine particles collected on the filtration membrane is digitalized by a detector 25. Thus, nickel and copper can be quantified.

FIG. 17 shows a case using a filtration membrane having a shape different from that shown in FIG. 16. The used filtration membrane has a turret shape as shown in FIG. 19 and filters through filter portions 26. A protection film 27 has portions other than the filter portions 26 bored to have a circle to prevent the liquid sample from leaking.

The turret-type filtration membrane of FIG. 19 corresponds to reference numeral 28 in FIG. 17 and has a structure that only the center of the turret-type filtration membrane 28 is fixed for rotation like a record.

The liquid sample is introduced through a sample introduction port 29. A cylinder 30 and a liquid sample discharge funnel 17 have the turret-type filtration membrane 25 between them, the next filter portion is aligned with the sample introduction port, and the next liquid sample passes through the filter portion. The filter portion through the filtration is rotates, so that the next new filter portion is aligned with the next sample introduction port, and the next liquid sample is filtered.

The liquid sample passes through the liquid sample discharge funnel 20 and drained into the drainage tank 21. If necessary, the drainage is discharged through the drainage line 22.

The filtering step is performed by suction filtration using the vacuum pump 23, and a suction force is adjusted by means of the cock 24.

The liquid sample is filtered, and the amount of fine particles collected on the filtration membrane is digitalized by the detector 25. Thus, nickel and copper can be quantified.

The example that FIG. 16 or FIG. 17 is shown at a terminal of the FIA shown in FIG. 15 was described above, but it is also possible to flow manually the liquid sample into the sample introduction portions of FIG. 16 and FIG. 17.

### BRIEF DESCRIPTION OF THE DRAWINGS

FIGS. 1A, 1B are views showing coloring of membrane filters involved in a change in nickel concentration.

FIGS. 2A, 2B are diagrams showing a relationship between a nickel concentration and reflection absorption of filtration membranes.

FIG. 3 is a view showing a photograph of filtration membranes sandwiched between slide glasses.

FIG. 4 is a diagram showing a relationship among nickel concentrations, copper concentrations and apparent light absorption.

FIG. 5 is a diagram showing a photograph of filtration membranes sandwiched between slide glasses.

FIG. 6 is a diagram showing a relationship among nickel concentration, copper concentration and apparent light absorption.

FIG. 7 is a view showing a photograph of filtration membranes sandwiched between slide glasses.

FIGS. 8A, 8B are diagrams showing a relationship between nickel and copper concentrations and apparent light absorption.

FIG. 9 is a diagram showing a relationship between nickel concentrations and average particle diameters of fine particles calculated from scattering intensity.

FIG. 10 is a view showing coloring of filtration membranes when the present method is applied to samples pretreated by solvent extraction.

FIGS. 11A, 11B are views showing coloring of filtration membranes in the coexistence of 50 ppb of copper.

FIG. 12 is a diagram showing a relationship between nickel concentrations and reflection absorption.

FIG. 13 is a view showing coloring of filtration membranes when the present method is applied to samples pretreated by solid-phase extraction.

FIG. 14 is a view showing coloring of filtration membranes when the present method is applied to samples pretreated by solid-phase extraction.

FIG. 15 is a diagram schematically showing an example of the structure of an apparatus used for a method for quantitative determination of nickel and copper according to the invention.

FIG. 16 is a diagram showing an example of the structure of a quantitative determination apparatus for nickel and copper.

FIG. 17 is a diagram showing an example of the structure of a quantitative determination apparatus for nickel and copper.

FIG. 18 is a diagram schematically showing a roll of filtration membrane.

FIG. 19 is a diagram schematically showing a turret-type filtration membrane.

### BEST MODE FOR CARRYING OUT THE INVENTION

Embodiments of the present invention will be described below with reference to the drawings.

### [Visual Colorimetric Determination of Trace Nickel]

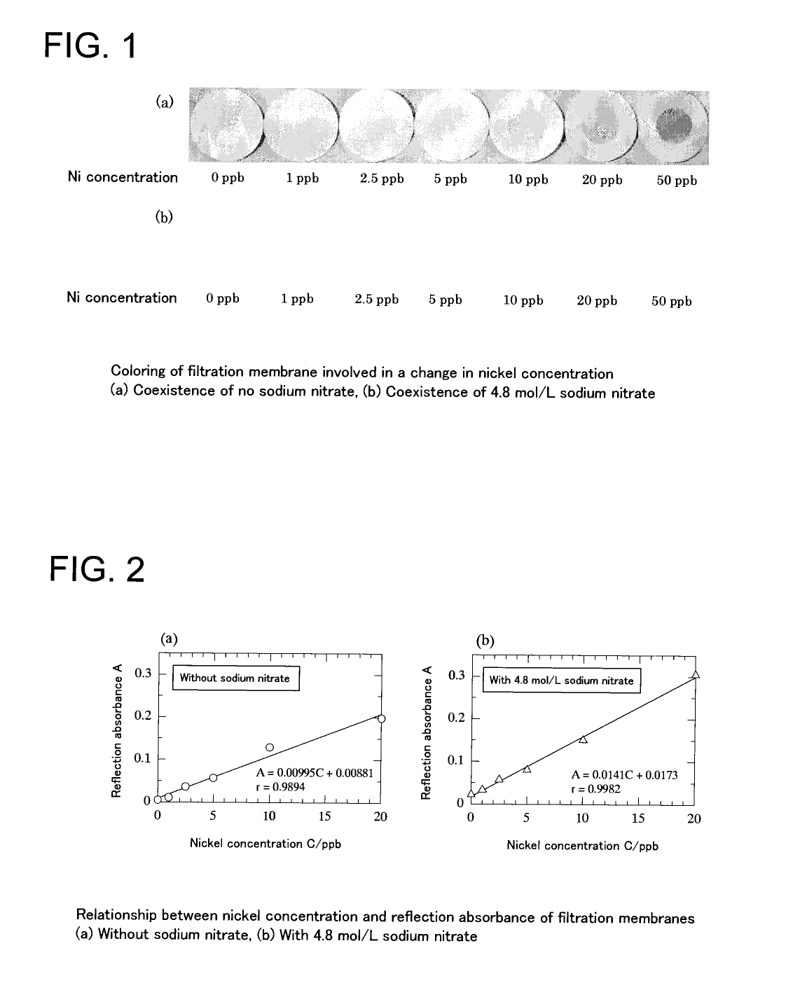

A 20 mL portion of the sample solution containing nickel ion and 6 mol/L sodium nitrate as a salting-out agent was taken into a Teflon (registered trademark) beaker, 2 ml of 0.63 mol/L sodium thiosulfate solution, 1 mL of 4.5×10⁻³ mol/L α-furildioxime (Tokyo Chemical Industry Co., Ltd.)/ethanol solution and 1.5 mL of pH buffer solution (0.1 mol/L TAPS-NaOH, pH 8.3, Wako Pure Chemical Industries, Ltd.) were added and diluted to 25 mL with water. The mixture was passed through a cellulose acetate type membrane filter (ADVANTEC, pore size 0.20 μm). Then, the filter was removed and dried at room temperature. The color transition of the membrane filters with relation to nickel content was observed by visual comparison. The nickel concentration was determined by visual comparison of the filter color shown in FIG. 1. It is preferable when 4.8 mol/L sodium nitrate coexists because coloring can be observed to be slightly darker in comparison with the case that it is not coexisted and it becomes easy to judge. In any case, the nickel concentration at the parts per billion levels was determined by visual comparison of the filter color.

### [Reflection Spectrometric Determination of Trace Nickel]

The color intensity of the filter which was prepared as mentioned above was estimated by reflection spectrometric measurement at 480 nm with a tristimulus colorimeter (model NF-777, NIPPON DENSHOKU INDUSTRIES CO., LTD.). The linear relationship between nickel content and signal intensity (the reflection spectrometric response) was obtained as shown in FIG. 2, and highly sensitive determination at parts per billion orders was possible.
It is also seen from the measurement that accurate quantitative determination of concentration is facilitated because the measurement in the coexistence of sodium nitrate increases a degree of change in reflection absorption involved in a change in nickel concentration.

### [Visual Simultaneous Determination of Nickel and Copper]

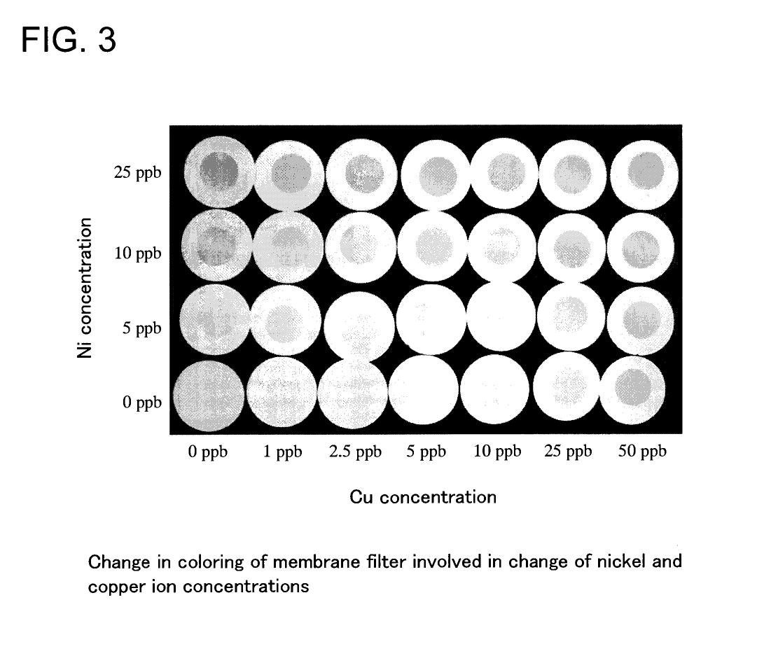

A sample solution containing 0-25 ppb of nickel and 0-50 ppb of copper was taken into a Teflon (registered trademark) beaker, a sodium nitrate solution, 2 mL of 4.5×10⁻³ mol/L α-furildioxime (Tokyo Chemical Industry Co., Ltd.)/ethanol solution, and 3 mL of pH buffer solution (0.1 mol/L, TAPS-NaOH pH 8.3, Wako Pure Chemical Industries, Ltd.) were added and diluted to 50 mL with water. The mixture was passed through a cellulose acetate type membrane filter (ADVANTEC, pore size 0.20 μm). Then, the filter was removed and dried at room temperature. The color transition of the membrane filters with relation to nickel and copper content was shown in FIG. 3. The nickel and copper concentration were determined simultaneously by visual comparison of the filter color.

### [Simultaneous Determination of Nickel and Copper by Tristimulus Colorimety]

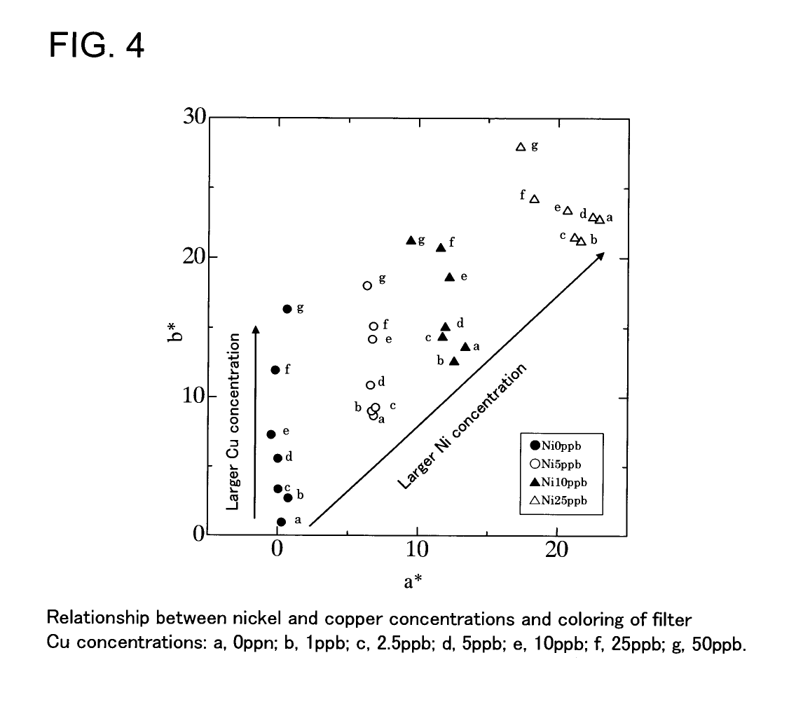

The chromaticity co-ordinates a* and b* of the colored filter which was prepared as mentioned above was estimated with a tristimulus colorimeter (model NF-777, NIPPON DENSHOKU INDUSTRIES CO., LTD.). The chromatic plots of the filter colors on an a*-b* diagram was shown in FIG. 4. It was found that the individual combinations of nickel and copper concentrations were reflected as the combinations of a* and b* values on the diagram, and nickel and copper could be determined simultaneously.

### [Quantitative Determination of Nickel and Copper by Sensor Configured of Light-Emitting Diode and Light-Receiving Element]

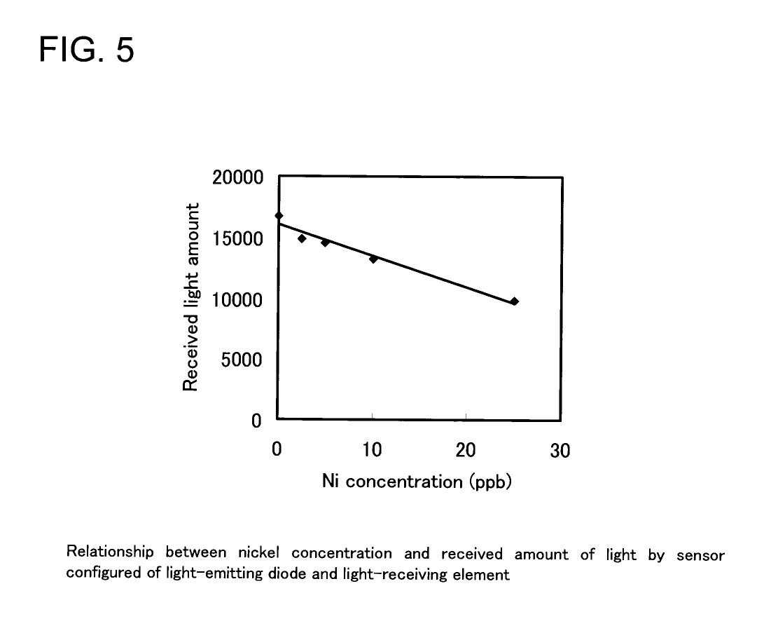

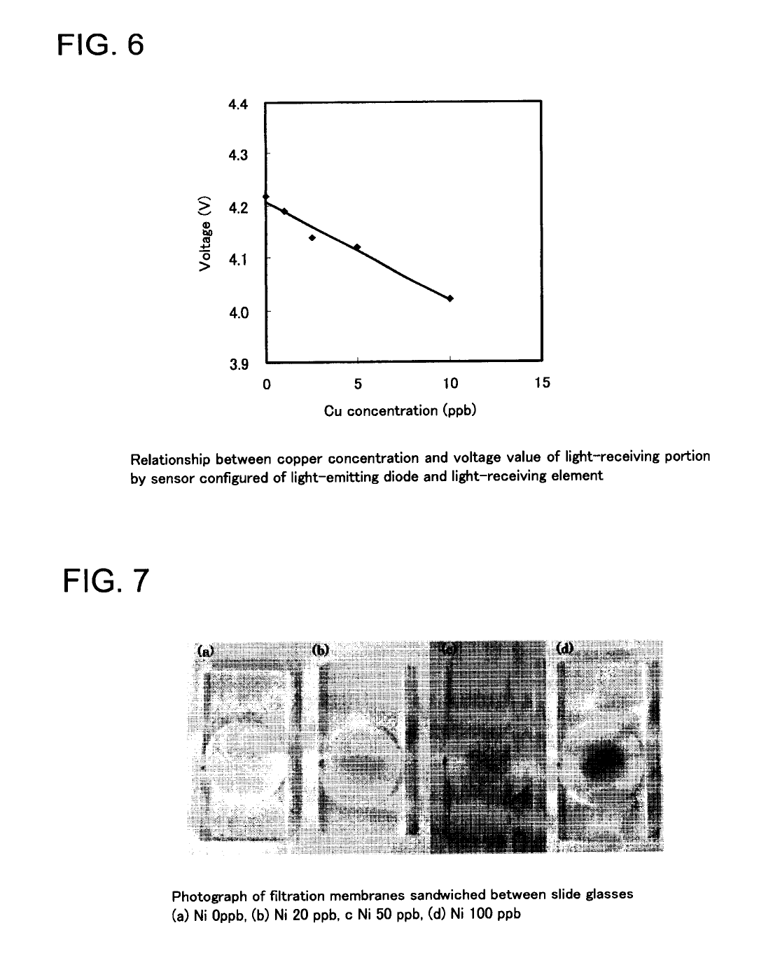

Colored degrees of the filtration membranes produced by the above-described operation were analyzed by a color sensor (manufactured by KEYENCE CORPORATION, a digital R•G•B sensor CZ-H35S) for nickel, a 370-nm ultraviolet LED (manufactured by Nichia Corporation, NSHU550; 1 mW) as a light emission source for copper, and a photodiode (manufactured by Hamamatsu Photonics K.K., S2386-18K) as a detection portion. As a result, first correlation was acknowledged between the nickel and copper concentrations and the received amount of reflected light, and highly sensitive quantitative determination on a ppb order was possible (FIGS. 5, 6).

### [Quantitative Determination of Nickel and Copper by Transmission Spectrometry]

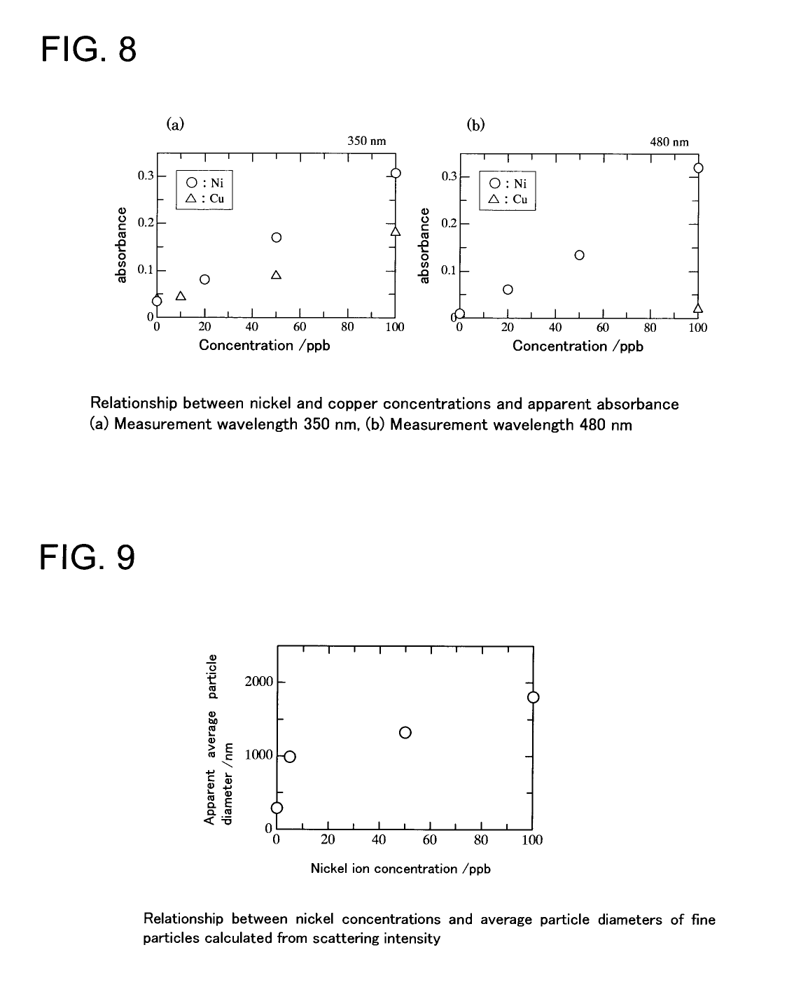

A 20 mL portion of the sample solution containing nickel ion and copper ion was taken into a Teflon (registered trademark) beaker, 1 mL of 4.5×10⁻³ mol/L α-furildioxime/ethanol solution and 1.5 mL of pH buffer solution (0.1 mol/L TAPS-NaOH, pH 8.3) were added and diluted to 25 mL with water. The mixture was passed through a Nuclepore membrane filter (Nomura Micro Science Co., Ltd., pore size 0.40 μm) which was conditioned prior to the filtration step by rinsing with ethanol. A spectrophotometer (JASCO Corporation, UVIDEC-210) was used to estimate the apparent absorbance values of the filters. The filter in a wet state (wetted with water if the filter was dry) was sandwiched between two sheets of glass plate (FIG. 7) and fixed on an edge in a sample chamber. The apparent absorbance values were measured at 350 and 480 nm against an unused filter. The results were shown in FIG. 8.

Linear relationships between the apparent absorbance value and concentration were observed in both copper and nickel, respectively. Therefore, the simultaneous determination of copper and nickel was enabled by the use of the above two wavelength in the transmission spectrometry.

### [Quantitative Determination of Nickel by Dynamic Light Scattering Measurement]

A 20 mL of sample solution containing nickel and 6 mol/L sodium nitrate was taken into a Teflon (registered trademark) beaker, 1 mL of 4.5×10⁻³ mol/L α-furildioxime/ethanol solution and 1.5 mL of pH buffer solution (0.1 mol/L TAPS-NaOH, pH 8.3) were added. The mixture was transferred to a volumetric flask made of polypropylene, and was filled up to 25 mL with water. Dynamic light scattering measurements of the solutions were performed using dynamic light scattering photometer (Otsuka Electronics Co., Ltd., DLS-7000). As a result, fine particles were observed in the solution, and a positive correlation was found between nickel concentration and apparent particle size, which was calculated by dynamic light scattering (see FIG. 9).

### [Observation of the Filter Surfaces by Scanning Electron Microscopy]

A 20 mL portion of the sample solution containing nickel ion was taken into a Teflon (registered trademark) beaker, 1 mL of 4.5×10⁻³ mol/L α-furildioxime/ethanol solution and 1.5 mL of pH buffer solution (0.1 mol/L TAPS-NaOH, pH 8.3) were added and diluted to 25 mL with water. The mixture was passed through a cellulose acetate type membrane filter (ADVANTEC, pore size 0.20 μm). Then, the filter was removed and dried at room temperature. A scanning electron microscope (JEOL DATUM LTD., JSM-5200) was used for observations of the filter surfaces. The fine particles were confirmed on the filter fiber by the scanning electron microscope observations, and the amount of the particles increased as the nickel concentration rose. The same phenomenon was also observed under the coexistence of 4.8 mol/L sodium nitrate. For comparison, the surfaces of the filters through which only water or a 4.8 mol/L sodium nitrate solution was passed were also observed in the same manner. These filters had substantially no change in comparison with the unused one. These results show that the nickel complex is collected as fine particulate matters on the filter.

### [Pretreatment of Liquid Sample by Solvent Extraction]

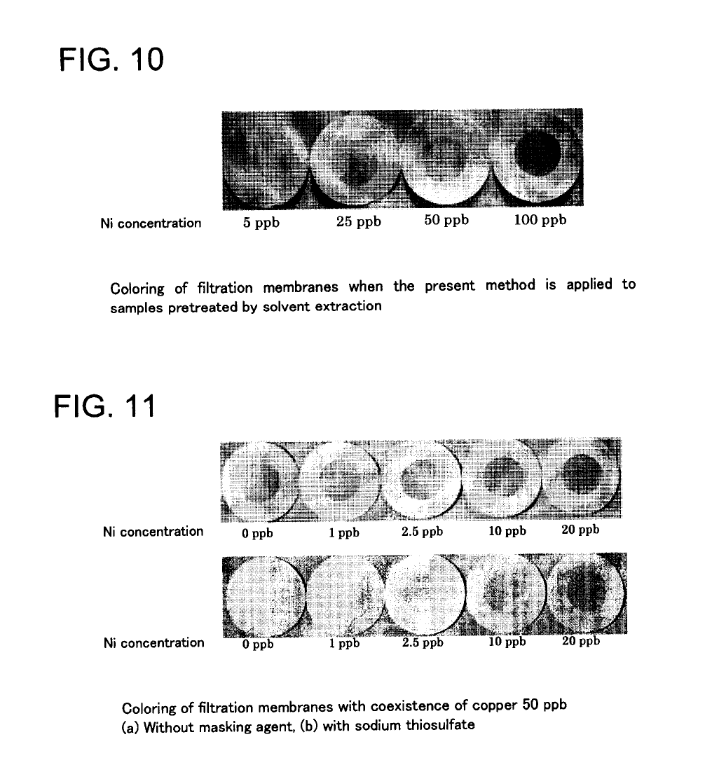

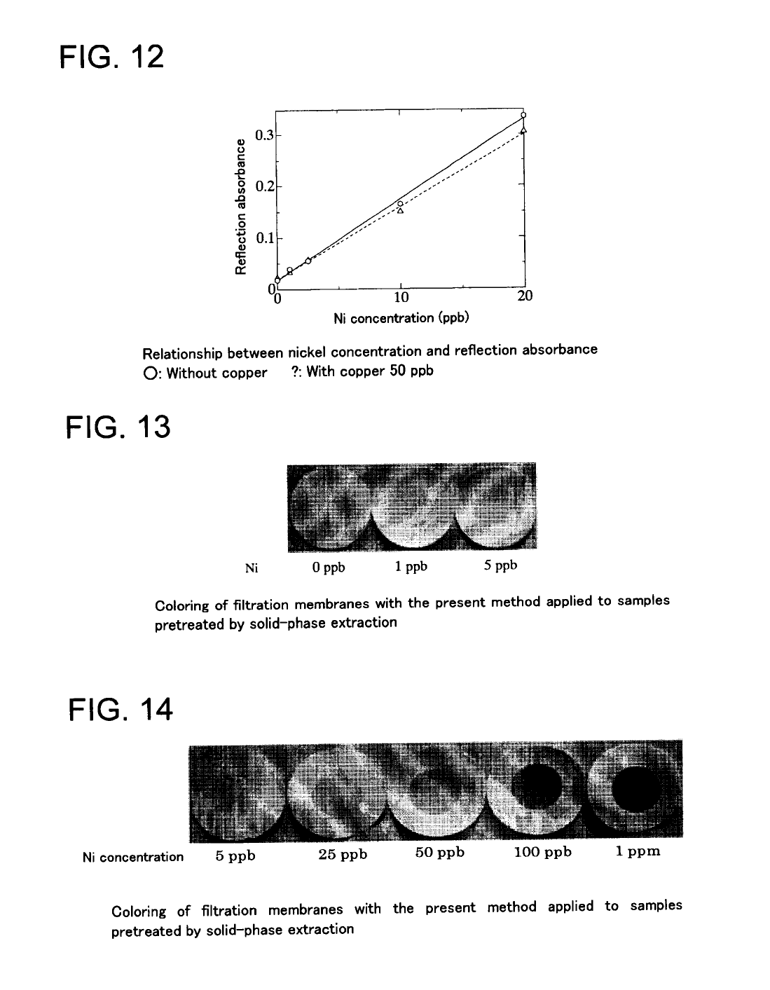

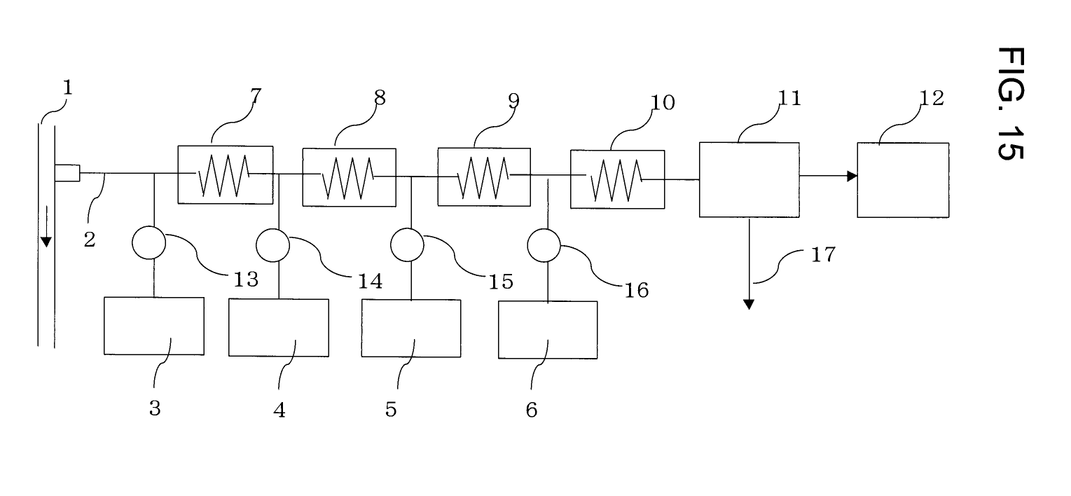

It was checked whether a pretreatment method of a sample by solvent extraction can be applied to the proposed method. Here, nickel was extracted into chloroform according to the procedure described in JIS K0101, and back-extraction into hydrochloric acid solution was performed. To 50 mL of a sample solution containing nickel, 2.5 mL of 100 g/L aqueous ammonium dicitrate solution and a phenolphthalein solution were added. Aqueous ammonia (1+5) was added until the solution was slightly red-colored, and 1 mL of 1% dimethylglyoxime/ethanol solution and 5 mL of chloroform were added, and the mixture was vigorously shaken for one minute. After standing the mixture at room temperature, the chloroform layer was transferred into another separating funnel. 3 mL of chloroform was added to a water layer, and the mixture was vigorously shaken for one minute. After standing at room temperature, the chloroform layer was transferred to another separating funnel, and the same operation was repeated once again. Then, about 20 mL of aqueous ammonia (1+50) was added to the separating funnel containing the chloroform layer, and the mixture was vigorously shaken for 30 seconds. After standing, the chloroform layer was transferred to another separating funnel. In addition, 5 mL of hydrochloric acid (1+20) was added to the separating funnel containing the chloroform layer, and the mixture was vigorously shaken for one minute. After standing at room temperature, and the chloroform layer was transferred to another separating funnel. Again, 2.5 mL of hydrochloric acid (1+20) was added to the chloroform layer, and the back-extraction operation was repeated. Then, the chloroform layer was discarded; the water layer was added to the previous water layer, and diluted to 50 mL with water. A 25 mL portion of the solution was taken into a beaker, and neutralized with sodium hydroxide. And 2 mL of 4.5×10⁻³ mol/L α-furildioxime/ethanol solution and 3 mL of a pH buffer solution (0.1 mol/L, TAPS-NaOH, pH 8.3) were added, and diluted to 50 mL with water. The mixture was passed through a cellulose acetate type membrane filter (ADVANTEC, pore size 0.20 μm). Then, the filter was removed and dried at room temperature. As shown in FIG. 10, the differences in nickel concentrations are clearly shown as dark and light colors of the filter, and it was confirmed that the pretreatment step by the solvent extraction can be applied to the present method.
[Pretreatment of Liquid Sample with Masking Agents]
The effect of a masking agent in the application to salinity samples of the proposed method was studied. Here, the masking of copper ion under the coexistence of 6 mol/L sodium nitrate was studied. A 20 mL of sample solution containing nickel and 6 mol/L sodium nitrate was taken into a Teflon (registered trademark) beaker, and 2 ml of 0.63 mol/L sodium thiosulfate solution as a masking agent, 1 mL of 4.5×10⁻³ mol/L α-furildioxime/ethanol solution, and 1.5 mL of a pH buffer solution (0.1 mol/L, TAPS-NaOH, pH 8.3) were added, and diluted to 25 mL with water. The mixture was passed through a cellulose acetate type membrane filter (ADVANTEC, pore size 0.20 μm). Then, the filter was removed and dried at room temperature. The color transition of the membrane filters with relation to nickel content was observed by visual comparison. And, the color intensity of the filter was estimated by reflection spectrometric measurement at 480 nm with a tristimulus colorimeter (model NF-777, NIPPON DENSHOKU INDUSTRIES CO., LTD.). As shown in FIG. 11, it was clearly different in the coloration of the filters between presence and absence of the masking agent. The influence of the coexistence of 50 ppb of copper was eliminated even in the sodium nitrate solution (see FIG. 12).

### [Pretreatment of Liquid Sample by Solid-Phase Extraction]

It was checked whether a pretreatment method of a sample by solid-phase extraction could be applied to the proposed method. Here, a pretreatment method of separating matrices was studied by solid-phase extraction of nickel using a chelating resin column (NOBIAS CHELATE-PA1, Hitachi High-Technologies Corporation) and by back-extraction into 3 mol/L of nitric acid. A 50 mL of a liquid sample containing nickel was passed through the column which was conditioned by a predetermined method. After washing with water, 8 mL of 3 mol/L nitric acid was passed through the column to recover nickel. After the eluate was neutralized by adding a sodium hydroxide solution, 2 mL of 4.5×10⁻³ mol/L α-furildioxime/ethanol solution and 3 mL of a pH buffer solution (0.1 mol/L, TAPS-NaOH, pH 8.3) were added, and diluted to 25 mL with water. The mixture was passed through a cellulose acetate type membrane filter (ADVANTEC, pore size 0.20 μm). Then, the filter was removed and dried at room temperature. As shown in FIG. 13, the differences in nickel concentrations were clearly shown as dark and light colors of the filter, and it was confirmed that the pretreatment method by the solid-phase extraction could be applied to the present method. It was also confirmed that the present solid-phase extraction method was effective as a pretreatment method when fine particulate matters coexisted in the solution.

### [Quantitative Determination of Nickel by Quantitative Determination Apparatus for Nickel and Copper]

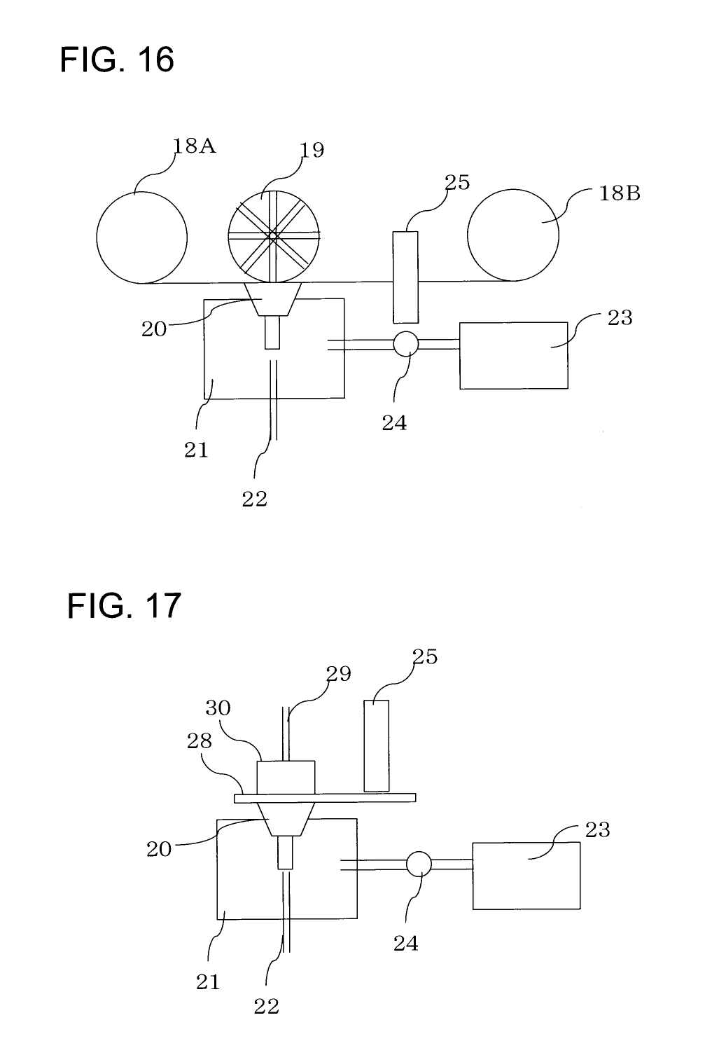

Based on the above basic study, the automated quantitative determination of nickel and copper was performed by using the apparatus shown in FIG. 15 and FIG. 16. A liquid sample containing 6 mol/L of sodium nitrate and 3 μg/L of nickel was flowing in the line 1. The liquid sample was collected at a flow rate of 1 mL/min from the sample pipe 2. In the reaction vessel 6, the pH value of the liquid sample was adjusted in the range of 8-9 by mixing the pH buffer solution (0.1 mol/L TAPS-NaOH (pH 9)), which was introduced with the pump 12 at a flow rate of 0.1 mL/min from the pH adjuster tank 3. Then, an aqueous solution of 0.63 mol/L sodium thiosulfate as a masking agent was introduced with the pump 13 at a flow rate of 0.1 mL/min from the masking agent tank 4, and mixed with the liquid sample in the reaction vessel 7. The complex-forming agent solution (4.5×10⁻³ mol/L α-furildioxime/ethanol solution), which was introduced with the pump 14 at a flow rate of 0.1 mL/min from the complex-forming agent tank, was mixed with the liquid sample in the reaction vessel 8, and the colored nickel complex which was fine particular state was formed.

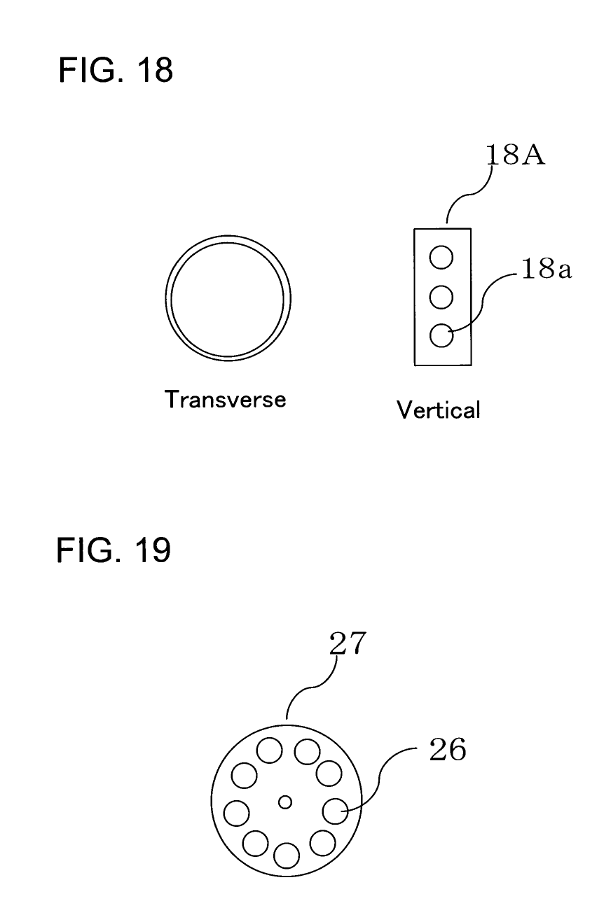

A prescribed amount of the mixture containing the nickel complex was flown into a sample injector 19 of the quantitative determination apparatus shown in FIG. 16 for nickel and copper, the mixture was passed through the filtration part of the roll-shape membrane filter shown in FIG. 18 under suction using a vacuum pump 23. The filtration part through which the mixture had been passed was sent to the detector 25 by rotating the sample injector.

For the analysis of the filtration part, a reflection spectrophotometer and a transmission spectrophotometer were used as detectors. For comparison, the liquid sample was also analyzed with an inductively coupled plasma mass spectrometer (ICP-MS, PerkinElmer Inc., ELAN (registered trademark) DRC II) The analytical results were summarized in Table 1. And, the results of the same test using a dynamic light scattering photometer, which was disposed at upstream of the filter of the flow injection analysis (FIA) apparatus shown in FIG. 15, are also shown in Table 1.

TABLE 1

| | | | | Dynamic |
|---|---|---|---|---|
| | | Reflection | Transmission | light |
| | ICP- | spectrometric | spectrometric | scattering |
| | MS | measurement | measurement | measurement |
| Nickel | 2.9 | 2.7 | 3.2 | 3.3 |
| concentration | | | | |
| (ppb) | | | | |

As shown in Table 1, substantially the same values as the results of the analysis using the ICP-MS were obtained. And, analysis of copper is also possible by selection of an appropriate masking agent. It was shown in the embodiment that the apparatus of the invention can omit some troublesome pre-treatments, such as desalting, neutralizing, concentration and the like involved in a conventional ICP-MS analysis method and can perform highly sensitive determination in a short time.

### INDUSTRIAL APPLICABILITY

The present invention can be used extensively for determination of nickel and/or copper which contain as metal impurities in a liquid sample.

What is claimed is:

1. A method for quantitative determination of nickel and/or copper, comprising:
forming colored fine particles of a nickel complex and/or a copper complex by adding a complex-forming agent which forms a complex with nickel and copper to a liquid sample containing unknown concentrations of nickel and/or copper; and
quantifying nickel and/or copper from the colored fine particles of nickel complex and/or copper complex.

2. The method for quantitative determination of nickel and/or copper according to claim 1, further comprising,
removing impure fine particulate matters contained in the liquid sample by the filtration membrane before the step of forming the colored fine particles of a nickel complex and/or a copper complex.

3. The method for quantitative determination of nickel and/or copper according to claim 1, further comprising,
adjusting pH by adding a chemical solution to the liquid sample before the step of forming the colored fine particles of a nickel complex and/or a copper complex.

4. The method for quantitative determination of nickel and/or copper according to claim 1, further comprising,
contacting the liquid sample to solids to adsorb nickel and/or copper onto the solids to condensate, removing components other than nickel and/or copper contained in the liquid sample, and eluting nickel and/or copper before the step of forming the colored fine particles of a nickel complex and/or a copper complex.

5. The method for quantitative determination of nickel and/or copper according to claim 1, further comprising,
extracting nickel and/or copper into a solvent which is not mixed with the liquid sample to condensate, removing components other than nickel and/or copper contained in the liquid sample and reextracting nickel and/or copper into an aqueous solution before the step of forming the colored fine particles of a nickel complex and/or a copper complex.

6. The method for quantitative determination of nickel and/or copper according to claim 1, further comprising,
suppressing interference by coexisting metals other than the nickel and/or copper by adding a masking agent to the liquid sample before the step of forming the colored fine particles of a nickel complex and/or a copper complex.

7. The method for quantitative determination of nickel and/or copper according to claim 1, further comprising,
adding a salting-out agent to the liquid sample to decrease the solubility of nickel and copper complexes and to liberate the fine particles of the complexes before forming the colored fine particles of nickel complex and/or copper complex.

8. The method for quantitative determination of nickel and/or copper according to claim 1,
wherein the step of quantifying nickel and/or copper from the colored fine particles of nickel complex and/or copper complex is performed by passing a liquid sample in which the fine particles of nickel complex and/or copper complex are formed through a filtration membrane to capture the fine particles of nickel complex and/or copper complex, and determining the colored degree of the colored filtration membrane by colorimetry.

9. The method for quantitative determination of nickel and/or copper according to claim 8,
wherein the filtration membrane is at least one selected from a reverse osmosis membrane, an ultrafiltration membrane and a microfiltration membrane.

10. The method for quantitative determination of nickel and/or copper according to claim 8,
wherein the filtration membrane has a shape which is at least one type selected from a flat membrane type, a roll type and a turret type.

11. The method for quantitative determination of nickel and/or copper according to claim 8,
wherein colorimetry of the colored degree of the colored filtration membrane is performed by comparing colors visually.

12. The method for quantitative determination of nickel and/or copper according to claim 8,
wherein colorimetry of the colored degree of the colored filtration membrane is determined by digitalizing by a spectrophotometer.

13. The method for quantitative determination of nickel and/or copper according to claim 8,
wherein colorimetry of the colored degree of the colored filtration membrane is determined by digitalizing by a sensor which is comprised of a light-emitting diode and a light-receiving element.

14. The method for quantitative determination of nickel and/or copper according to claim 1,
wherein the step of quantifying nickel and/or copper from the colored fine particles of a nickel complex and/or a copper complex is performed by calculating from the number of particles and/or particle diameters of the fine particles of the nickel complex and/or the copper complex by light scattering intensity.

15. A quantitative determination apparatus for nickel and/or copper, comprising:
a sample pipe to collect a liquid sample from a liquid sample line including unknown concentrations of nickel and/or copper at predetermined time intervals;
a complex-forming agent storing tank to store a complex-forming agent which forms a complex with nickel and copper contained in the liquid sample;
a reaction vessel to form colored fine particles of a nickel complex and/or a copper complex by reacting the complex-forming agent fed from the complex-forming agent storing tank with the liquid sample fed from the sample pipe;
a filter having a filtration membrane to capture the colored fine particles of a nickel complex and/or a copper complex fed from the reaction vessel; and
a quantitative determination means to quantify nickel and copper from the nickel complex and/or the copper complex captured by the filtration membrane.

16. A quantitative determination apparatus for nickel and/or copper, comprising:
a sample pipe to collect a liquid sample from a liquid sample line including unknown concentrations of nickel and/or copper at predetermined time intervals;
a complex-forming agent storing tank to store a complex-forming agent to form a complex with nickel and copper contained in the liquid sample;
a reaction vessel to form colored fine particles of a nickel complex and/or a copper complex by reacting the complex-forming agent fed from the complex-forming agent storing tank with the liquid sample fed from the sample pipe; and
a quantitative determination means to quantify the colored fine particles of a nickel complex and/or a copper complex fed from the reaction vessel.
